(19) United States
(12) Patent Application Publication
(10) Pub. No.: US 2011/0201918 A1
(43) Pub. Date: Aug. 18, 2011

(54) Radiotherapy and imaging apparatus

(75) Inventor: Duncan Neil Bourne, Surrey (GB)

(73) Assignee: Elekta AB (Publ), Stockholm (SE)

(21) Appl. No.: 12/704,944

(22) Filed: Feb. 12, 2010

(51) Int. Cl. A61B 5/055; A61N 5/00; G21K 1/04

(52) U.S. Cl. 600/411; 250/492.1; 250/505.1

(57) ABSTRACT

A radiotherapy system comprises a patient support, moveable along a translation axis, an imaging apparatus, comprising a first magnetic coil and a second magnetic coil, the first and second magnetic coils having a common central axis parallel to the translation axis, and being displaced from one another along the central axis to form a gap therebetween, the imaging apparatus being configured to obtain an image of a patient on the patient support and a source of radiation mounted on a chassis, the chassis being rotatable about the central axis and the source being adapted to emit a beam of radiation through the gap along a beam axis that intersects with the central axis, the beam having a first extent in a first direction parallel to the central axis, and a second, greater extent in a second direction transverse to the central axis.

### FIELD OF THE INVENTION

The present invention relates to radiotherapy apparatus, and particularly to a radiotherapy apparatus comprising a magnetic resonance imaging (MRI) apparatus.

### BACKGROUND ART

It is known that exposure of human or animal tissue to ionising radiation will kill the cells thus exposed. This finds application in the treatment of pathological cells, for example. In order to treat tumours deep within the body of the patient, the radiation must however penetrate the healthy tissue in order to irradiate and destroy the pathological cells. In conventional radiation therapy, large volumes of healthy tissue can thus be exposed to harmful doses of radiation, resulting in prolonged recovery periods for the patient. It is, therefore, desirable to design a device for treating a patient with ionising radiation and treatment protocols so as to expose the pathological tissue to a dose of radiation which will result in the death of those cells, whilst keeping the exposure of healthy tissue to a minimum.

Several methods have previously been employed to achieve the desired pathological cell-destroying exposure whilst keeping the exposure of healthy cells to a minimum. Many methods work by directing radiation at a tumour from a number of directions, either simultaneously from multiple sources or multiple exposures from a single source. The intensity of radiation emanating from each direction is therefore less than would be required to actually destroy cells (although still sufficient to damage the cells), but where the radiation beams from the multiple directions converge, the intensity of radiation is sufficient to deliver a therapeutic dose. By providing radiation from multiple directions, the amount of radiation delivered to surrounding healthy cells can be minimized.

The shape of the beam varies. For single-source devices, cone beams centred on the isocentre are common, while fan beams are also employed (for example as shown in U.S. Pat. No. 5,317,616).

Of course it is also important that the radiation should be accurately targeted on the region that requires treatment. For this reason, patients are required to remain still for the duration of the therapy session, to minimize the risk of damage to healthy tissue surrounding the target region. However, some movement is inevitable, e.g. through breathing, or other involuntary movements.

To overcome this problem, it is known to integrate an image acquisition system with the radiotherapy apparatus, to provide real-time imaging of the region and ensure that the radiation emitted by the radiotherapy apparatus tracks any movement of the patient. However, the choice of imaging system is in general limited by the radiotherapy apparatus in which it is installed, and in particular by the geometry. For example, magnetic resonance imaging (MRI) systems require magnetic coils to be placed around the patient. However, these coils will act to block therapeutic radiation from reaching the patient.

What is required is an integrated radiotherapy system that delivers high-quality in both the imaging and treatment of a patient.

### SUMMARY OF THE INVENTION

The inventors of the present invention have overcome the problems associated with conventional integrated radiotherapy and imaging systems, by providing a radiotherapy system integrating a fan-beam based radiation source with an MRI apparatus. Instead of a single coil generating a magnetic field, two coils are spaced slightly apart creating a narrow gap or window through which radiation may be imparted to the patient. By integrating such an MRI apparatus with a fan-beam source of radiation, the two coils may be placed closer together than with conventional systems, allowing higher magnetic fields and increasing the quality of MRI images without adversely affecting the quality of treatment supplied to the patient.

The present invention therefore provides, according to one aspect, a radiotherapy system comprising a patient support, moveable along a translation axis, an imaging apparatus, comprising a first magnetic coil and a second magnetic coil, the first and second magnetic coils having a common central axis parallel to the translation axis, and being displaced from one another along the central axis to form a gap therebetween, the imaging apparatus being configured to obtain an image of a patient on the patient support and a source of radiation mounted on a chassis, the chassis being rotatable about the central axis and the source being adapted to emit a beam of radiation through the gap along a beam axis that intersects with the central axis, the beam having a first extent in a first direction parallel to the central axis, and a second, greater extent in a second direction transverse to the central axis.

In one embodiment, the relatively narrow dimension is between about 2 cm and about 5 cm when projected on to the isocentric plane, and in another embodiment between about 2 cm and 3 cm when projected on to the isocentric plane. Previous designs (such as our international application WO2004/024235) have had large gaps in order to accommodate the cone-shaped radiation beam, whereas the system according to embodiments of the present invention could employ a gap as narrow as 2 cm (assuming that the beam does not broaden significantly in the dimension parallel to the central axis as it passes through the gap and on to the isocentric plane). Therefore whilst previous designs were limited to magnetic fields of the order of 1.5 T, the design according to embodiments of the present invention is able to increase the magnetic field strength to 3 T, for example, with corresponding improvements in image quality.

In an embodiment, the system further comprises a multi-leaf collimator comprising a plurality of elongate leaves disposed with their longitudinal directions substantially aligned with the first direction and movable in that direction to either a withdrawn position in which the leaf lies outside the beam, or an extended position in which the leaf projects across the beam. That is, the multi-leaf collimator may be a so-called “binary multi-leaf collimator”, in that the leaves can only occupy one of two positions.

The multi-leaf collimator disclosed above may comprise a respective plurality of pneumatic or hydraulic actuators, for moving the plurality of elongate leaves. Such actuators are not affected by the magnetic field generated by the MRI coils, and therefore it is possible that higher magnetic fields may be used.

In one embodiment, the chassis is continuously rotatable about the central axis. In this embodiment, the patient support may be configured to move along the translation axis as the chassis rotates about the central axis, resulting in a helical radiation delivery pattern. Such a pattern is known to produce high quality dose distributions.

In a further embodiment, the system further comprises a detector mounted to the chassis opposite the source.

### BRIEF DESCRIPTION OF THE DRAWINGS

An embodiment of the present invention will now be described by way of example, with reference to the accompanying figures in which;

FIG. 1 shows a radiotherapy system according to embodiments of the present invention.

FIG. 2 is a schematic diagram of aspects of the radiotherapy system according to embodiments of the present invention.

FIG. 3 shows a multi-leaf collimator according to an embodiment of the present invention.

FIG. 4 shows a multi-leaf collimator according to another embodiment of the present invention.

### DETAILED DESCRIPTION OF THE EMBODIMENTS

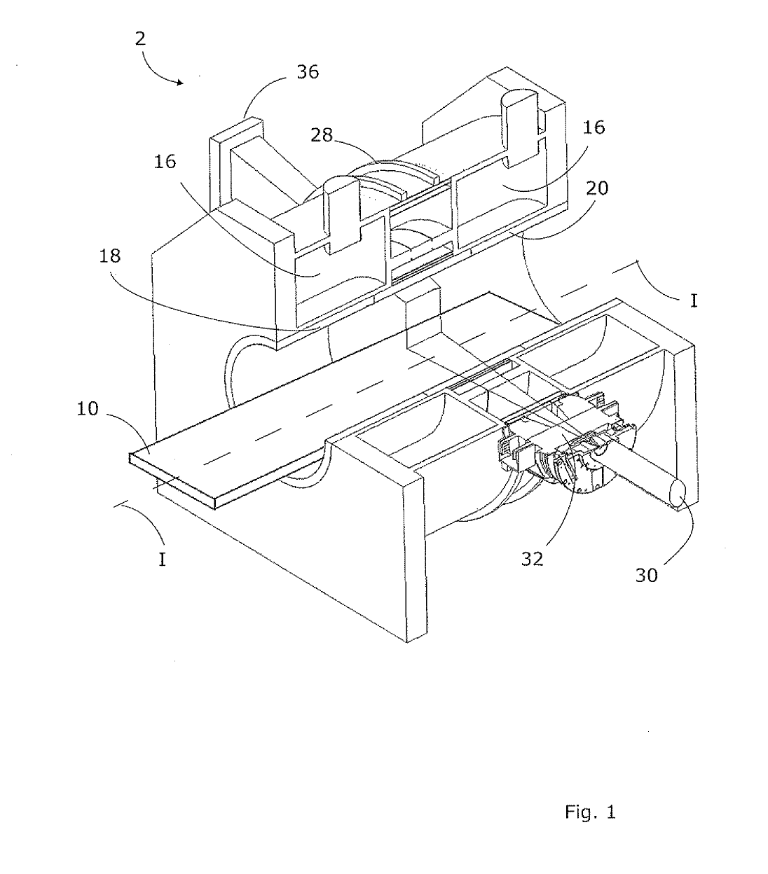

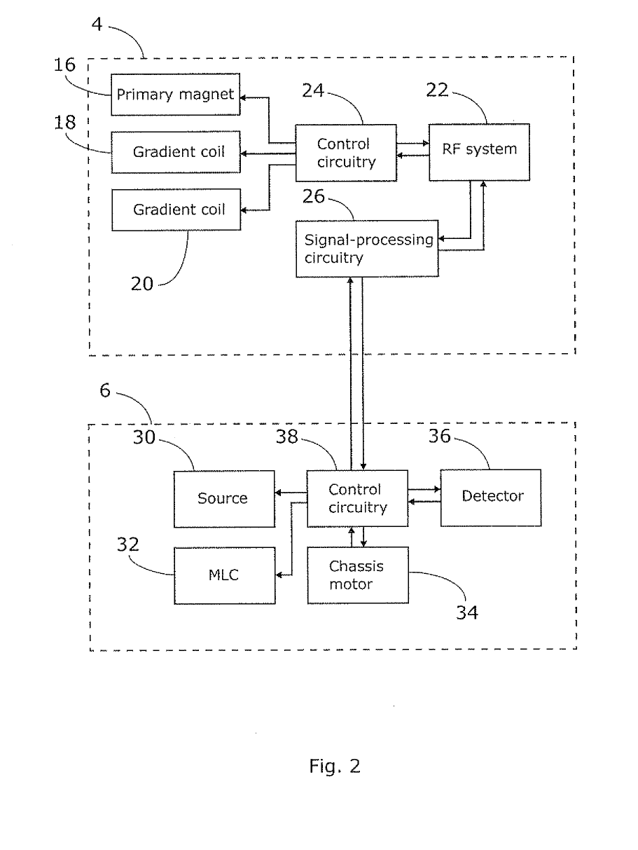

FIG. 1 shows a system according to embodiments of the present invention, comprising a radiotherapy apparatus and a magnetic resonance imaging (MRI) apparatus. The radiotherapy apparatus 6 and MRI apparatus 4 are shown schematically in FIG. 2.

The system includes a couch 10, for supporting a patient in the apparatus. The couch 10 is movable along a horizontal, translation axis (labelled “I”), such that a patient resting on the couch is moved into the radiotherapy and MRI apparatus. In one embodiment, the couch 10 is rotatable around a central vertical axis of rotation, transverse to the translation axis, although this is not illustrated. The couch 10 may form a cantilever section that projects away from a support structure (not illustrated). In one embodiment, the couch 10 is moved along the translation axis relative to the support structure in order to form the cantilever section, i.e. the cantilever section increases in length as the couch is moved and the lift remains stationary. In another embodiment, both the support structure and the couch 10 move along the translation axis, such that the cantilever section remains substantially constant in length, as described in our U.S. patent application Ser. No. 11/827,320 filed on 11 Jul. 2007.

As mentioned above, the system 2 also comprises an MRI apparatus 4, for producing real-time imaging of a patient positioned on the couch 10. The MRI apparatus includes a primary magnet 16 which acts to generate the so-called “primary” magnetic field for magnetic resonance imaging. That is, the magnetic field lines generated by operation of the magnet 16 run substantially parallel to the central translation axis I. The primary magnet 16 consists of one or more coils with an axis that runs parallel to the translation axis I. The one or more coils may be a single coil or a plurality of coaxial coils of different diameter, as illustrated. In one embodiment, the one or more coils in the primary magnet 16 are spaced such that a central window of the magnet 16 is free of coils. In other embodiments, the coils in the magnet 16 may simply be thin enough that they are substantially transparent to radiation of the wavelength generated by the radiotherapy apparatus. The magnet 16 may further comprise one or more active shielding coils, which generates a magnetic field outside the magnet 16 of approximately equal magnitude and opposite polarity to the external primary magnetic field. The more sensitive parts of the system 2, such as the accelerator, are positioned in this region outside the magnet 16 where the magnetic field is cancelled, at least to a first order. The MRI apparatus 4 further comprises two gradient coils 18, 20, which generate the so-called “gradient” magnetic field that is superposed on the primary magnetic field. These coils 18, 20 generate a gradient in the resultant magnetic field that allows spatial encoding of the protons so that their position can be determined from the frequency at which resonance occurs (the Larmor frequency). The gradient coils 18, 20 are positioned around a common central axis with the primary magnet 16, and are displaced from one another along that central axis. This displacement creates a gap, or window, between the two coils 18, 20. In one embodiment, the gap is between about 2 cm and about 5 cm, and in another embodiment between about 2 cm and 3 cm. In an embodiment where the primary magnet 16 also comprises a central window between coils, the two windows are aligned with one another.

An RF system 22 transmits radio signals at varying frequencies towards the patient, and detects the absorption at those frequencies so that the presence and location of protons in the patient can be determined. The RF system 22 may include a single coil that both transmits the radio signals and receives the reflected signals, dedicated transmitting and receiving coils, or multi-element phased array coils, for example. Control circuitry 24 controls the operation of the various coils 16, 18, 20 and the RF system 22, and signal-processing circuitry 26 receives the output of the RF system, generating therefrom images of the patient supported by the couch 10.

As mentioned above, the system 2 further comprises a radiotherapy apparatus 6 which delivers doses of radiation to a patient supported by the couch 10. The majority of the radiotherapy apparatus 6, including at least a source of radiation 30 (e.g. an x-ray source) and a multi-leaf collimator (MLC) 32, is mounted on a chassis 28. The chassis 28 is continuously rotatable around the couch 10 when it is inserted into the treatment area, powered by one or more chassis motors 34. In the illustrated embodiment, a radiation detector 36 is also mounted on the chassis 28 opposite the radiation source 30 and with the rotational axis of the chassis positioned between them. The radiotherapy apparatus 6 further comprises control circuitry 38, which may be integrated within the system 2 shown in FIG. 1 or remote from it, and controls the source the radiation source 30, the MLC 32 and the chassis motor 34.

The radiation source 30 is positioned to emit radiation through the window defined by the two gradient coils 18, 20, and also through the window defined in the primary magnet 16. According to embodiments of the present invention, the source 30 emits so-called “fan beams” of radiation. The radiation beam is collimated with appropriate shielding prior to arrival at the MLC 32, by which time it is already “letterbox-shaped” in order to pass through the MLC housing as described in greater detail below. That is, the radiation beam is relatively narrow in one dimension parallel to the axis of rotation of the chassis 28, and is relatively wide in a dimension that is transverse to the axis of rotation of the chassis. In one embodiment, the relatively narrow dimension is between about 2 cm and about 5 cm when projected on to the isocentric plane, and in another embodiment between about 2 cm and 3 cm when projected on to the isocentric plane. Thus, the beam takes the fan shape that gives it its name. It is this fan-shaped beam that is ideally suited to the geometry of the system 2, in which two gradient coils 18, 20 are displaced from one another in order to allow the radiation access to the patient. A fan-shaped beam provides substantial radiation to the patient through the narrow window, meaning that the gradient coils 18, 20 can be placed closer together than with conventional integrated radiotherapy/imaging systems. This allows the gradient coils 18, 20 to generate much stronger gradient fields than would otherwise be the case, increasing the quality of the images obtained by the MRI apparatus 4.

In operation, a patient is placed on the couch 10 and the couch is inserted into the treatment area defined by the magnetic coils 16, 18 and the chassis 28. The control circuitry 38 controls the radiation source 30, the MLC 32 and the chassis motor to deliver radiation to the patient through the window between the coils 16, 18. The control circuitry 38 controls the source to deliver radiation in a fan beam, in the usual pulsed manner. The chassis motor 34 is controlled such that the chassis 28 rotates about the patient, meaning the radiation can be delivered from different directions. The MLC 32 is controlled to take different shapes, thereby altering the shape of the beam as it will reach the patient. Simultaneously with rotation of the chassis 28 about the patient, the couch 10 may be moved along a translation axis into or out of the treatment area (i.e. parallel to the axis of rotation of the chassis). With this simultaneous motion a helical radiation delivery pattern is achieved, known to produce high quality dose distributions.

The MRI apparatus 4, and specifically the signal-processing circuitry 26, delivers real-time (or in practice near real-time, after a delay in the order of milliseconds) images of the patient to the control circuitry 38. This information allows the control circuitry to adapt the operation of the source 30, MLC 32 and/or chassis motor 34, such that the radiation delivered to the patient accurately tracks the motion of the patient, for example due to breathing.

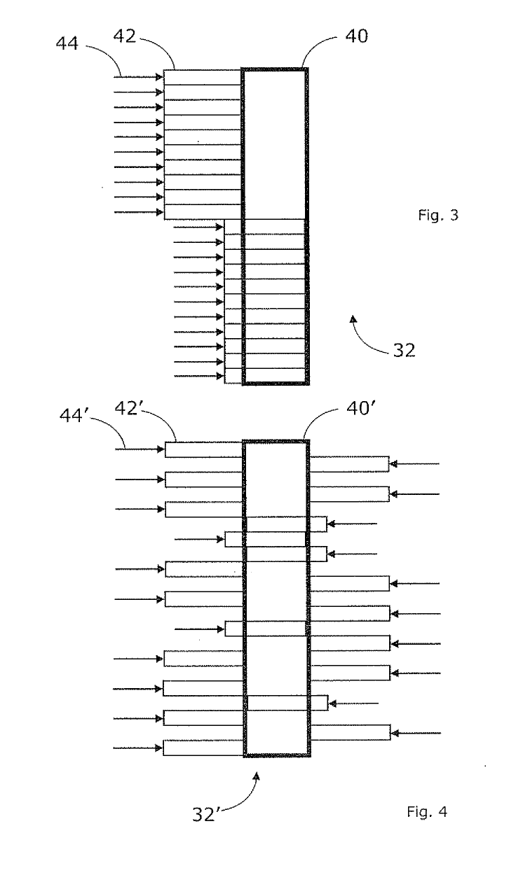

FIG. 3 shows an MLC 32 according to one embodiment of the present invention. The MLC comprises a housing 40 and a plurality of leaves 42 that slot into the housing. The MLC 32 also comprises a plurality of actuators 44, each actuator being coupled to a respective leaf 48. The housing 40 is effectively a slit through which radiation passes on its way to the patient. The leaves 42 move into and out of the slit in order to selectively block parts of the radiation from reaching the patient. In this embodiment the MLC 32 is a binary collimator, in that each leaf 42 is movable by action of the actuators 44 between two positions: a first position in which the leaf is completely inserted into the housing; and a second position in which the leaf is fully, or substantially fully retracted from the housing. In the first position, the portion of radiation defined by the leaf's position in the housing is blocked from reaching the patient; in the second position, that portion of radiation is allowed through the MLC 42 to the patient. In embodiments of the invention, the actuators may be pneumatic or hydraulic, such that they may operate with minimal interference from the strong magnetic fields created by the MRI apparatus 4.

In this embodiment, the shape of the field is not adjusted, but the time for which the leaves are opened is varied, thereby controlling the radiation fluence that passes though the slit. Due to the slit nature of the collimator, this is used in conjunction with longitudinal motion of the patient (i.e. along the translation axis) so as to cover the extent of the target transverse to the slit.

The leaves 42 may be thicker in parts further from the source of radiation 30 than parts nearer the source of radiation. That is, as the radiation beam diverges into the fan shape according to the present invention, so the leaves also increase in width so that the radiation beam is effectively blocked.

FIG. 4 shows an MLC 32′ according to another embodiment of the present invention. The MLC 32′ is substantially similar to the MLC 32 described with respect to FIG. 3, and so will not be described in great detail. Thus, the MLC 32′ comprises a slit housing 40′, and a plurality of leaves 42′ that are separately movable between two positions in which the leaves are completely inserted into the housing, or fully, or substantially fully retracted from the housing, as described above.

In this embodiment, however, the leaves 42′ are positioned on alternate sides of the housing 40′ when in their respective retracted positions. Similarly, the respective actuators 44′ are also positioned on alternate sides in order to actuate the leaves into and out of the housing. By placing the leaves in these alternating positions, the space constraints placed on each actuator 44′ are relaxed, as each actuator has twice as much room.

The present invention therefore provides a system which incorporates both a radiotherapy apparatus and an MRI apparatus. Both the radiotherapy apparatus and the MRI apparatus are adapted so that they can work together, while maintaining a high level of quality in their respective operations. The MRI apparatus is adapted to comprise a radiation-transmissive primary coil and two gradient coils that are spaced apart, creating a narrow window through which radiation may be delivered to the patient with minimal attenuation. The radiotherapy apparatus is adapted to deliver radiation in a fan beam, which makes the best use of the narrow window provided by the magnetic coils. By combining these two adaptations, a high level of radiation may be delivered to the patient, with high-quality imaging.

It will of course be understood that many variations may be made to the above-described embodiment without departing from the scope of the present invention.

Although the present invention has been described with reference to preferred embodiments, workers skilled in the art will recognize that changes may be made in form and detail without departing from the spirit and scope of the invention.

1. A radiotherapy system comprising:
a patient support, moveable along a translation axis;
an imaging apparatus, comprising a first magnetic coil and a second magnetic coil, the first and second magnetic coils having a common central axis parallel to the translation axis, and being displaced from one another along the central axis to form a gap therebetween, the imaging apparatus being configured to obtain an image of a patient on the patient support; and
a source of radiation mounted on a chassis, the chassis being rotatable about the central axis and the source being adapted to emit a beam of radiation through the gap along a beam axis that intersects with the central axis, the beam having a first extent in a first direction parallel to the central axis, and a second, greater extent in a second direction transverse to the central axis.

2. The radiotherapy system as claimed in claim 1, further comprising a multi-leaf collimator comprising a plurality of elongate leaves disposed with their longitudinal directions substantially aligned with the first direction and movable in that direction to either a fully withdrawn position in which the leaf lies outside the beam, or a fully extended position in which the leaf projects across the beam.

3. The radiotherapy system as claimed in claim 2, wherein adjacent leaves of the plurality of leaves are located on opposing sides of the beam when in their respective fully withdrawn positions.

4. The radiotherapy system as claimed in claim 2, wherein the multi-leaf collimator further comprises a respective plurality of pneumatic or hydraulic actuators, for moving the plurality of elongate leaves.

5. The radiotherapy system as claimed in claim 1, wherein the patient support is configured to move along the translation axis as the chassis rotates about the central axis, resulting in a helical radiation delivery pattern.

6. The radiotherapy system as claimed in claim 1, further comprising a control means for the source adapted to control the source so as to deliver a therapeutic radiation dose to a patient on the patient support, the control means being adapted to receive magnetic resonance images from the imaging apparatus during delivery of the dose.

7. The radiotherapy system as claimed in claim 1, in which the chassis is continuously rotatable about the central axis.

8. The radiotherapy system as claimed in claim 1, further comprising a radiation detector mounted to the chassis opposite the source.
